(19) United States
(12) Patent Application Publication
(10) Pub. No.: US 2017/0334396 A1
(43) Pub. Date: Nov. 23, 2017

(54) ANTI-THEFT METHOD AND DEVICE FOR A BALANCE VEHICLE AND COMPUTER-READABLE MEDIUM

(71) Applicant: Beijing Xiaomi Mobile Software Co., Ltd, Beijing (CN)

(72) Inventors: Shangquan LIN, Beijing (CN); Xing YONG, Beijing (CN); Xiaolin HA, Beijing (CN); Xiaotong ZHANG, Beijing (CN)

(73) Assignee: Beijing Xiaomi Mobile Software Co., Ltd, Beijing (CN)

(21) Appl. No.: 15/360,533

(22) Filed: Nov. 23, 2016

(30) Foreign Application Priority Data
May 23, 2016 (CN) 201610346479.3

(51) Int. Cl. B60R 25/25; A61B 5/103; A61B 5/117; A61B 5/00

(52) U.S. Cl. CPC B60R 25/25; A61B 5/742; A61B 5/103; A61B 5/117; B60R 2325/30

(57) ABSTRACT

The disclosure relates to an anti-theft method for a balance vehicle, a device, and a non-transitory computer readable medium. The method includes measuring a first set of physiological characteristics of a first user that is currently occupying the balance vehicle; retrieving a second set of physiological characteristics from storage, wherein the second set of physiological characteristics is associated with at least one second user that is permitted to operate the balance vehicle; comparing the first set of physiological characteristics with the second set of physiological characteristics; determining whether the first user is different from the second user based on the comparison; and generating a prompt that is configured to indicate whether the first user is different from the second user based on the determination.

### CROSS-REFERENCE TO RELATED APPLICATION

This application is based on and claims priority to Chinese Patent Application No. 201610346479.3 filed on May 23, 2016, which is incorporated herein by reference in its entirety.

### FIELD

The present disclosure generally relates to the field of balance vehicles, and more particularly to an anti-theft method and device for a balance vehicle and computer-readable medium.

### BACKGROUND

The balance vehicle is a personal transportation device for short distance riding instead of walking. The balance vehicle may alleviate the traffic pressure in the city to a certain extent, and may also be a popular element of street fashion. The balance vehicle is small and light. While these portable features may be its advantage, the balance vehicle may be easily stolen as a result.

### SUMMARY

This Summary is provided to introduce a selection of aspects of the present disclosure in a simplified form that are further described below in the Detailed Description. This Summary is not intended to identify key features or essential features of the claimed subject matter, nor is it intended to be used to limit the scope of the claimed subject matter.

Aspects of the disclosure provide an anti-theft method for a balance vehicle. The method includes measuring a first set of physiological characteristics of a first user that is currently occupying the balance vehicle; retrieving a second set of physiological characteristics from storage, wherein the second set of physiological characteristics is associated with at least one second user that is permitted to operate the balance vehicle; comparing the first set of physiological characteristics with the second set of physiological characteristics; determining whether the first user is different from the second user based on the comparison; and generating a prompt that is configured to indicate whether the first user is different from the second user based on the determination.

The first set of physiological characteristics includes at least one of a weight of the first user and an area occupied by a foot of the first user. The weight of the first user is measured via a weight sensor installed on the balance vehicle. The area is measured via a pressure sensor installed on the balance vehicle. The prompt is generated by at least one of displaying content and playing voice.

The method also includes receiving an update instruction with respect to the second set of physiological characteristics; and updating the second set of physiological characteristics based on the update instruction, wherein the update instruction includes at least one of an adding instruction, a deleting instruction, and a modifying instruction.

When the update instruction includes at least one of the adding instruction and the modifying instruction, updating the second set of physiological characteristics includes receiving physiological characteristics information based on the update instruction; and updating the second set of physiological characteristics based on the received physiological characteristics information.

The physiological characteristics information is received via a terminal or via the balance vehicle.

Comparing the first set of physiological characteristics with the second set of physiological characteristics includes determining whether the second set of physiological characteristics includes the first set of physiological characteristics. The method also includes disabling operation of the balance vehicle by the first user when it is determined that the second set of physiological characteristics does not include the first set of physiological characteristics; and enabling operation of the balance vehicle by the first user when it is determined that the second set of physiological characteristics includes the first set of physiological characteristics.

Aspects of the disclosure also provide an anti-theft device for a balance vehicle. The device includes a processor and a memory configured to store instructions executable by the processor. The processor is configured to measure a first set of physiological characteristics of a first user that is currently occupying the balance vehicle; retrieve a second set of physiological characteristics from the memory, wherein the second set of physiological characteristics is associated with at least one second user that is permitted to operate the balance vehicle; compare the first set of physiological characteristics with the second set of physiological characteristics; determine whether the first user is different from the second user based on the comparison; and generate a prompt that is configured to indicate whether the first user is different from the second user based on the determination.

The first set of physiological characteristics includes at least one of a weight of the first user and an area occupied by a foot of the first user. The weight of the first user is measured via a weight sensor installed on the balance vehicle. The area is measured via a pressure sensor installed on the balance vehicle. The processor is configured to generate the prompt by at least one of displaying content and playing voice.

The processor is also configured to receive an update instruction with respect to the second set of physiological characteristics; and update the second set of physiological characteristics based on the update instruction, wherein the update instruction includes at least one of an adding instruction, a deleting instruction, and a modifying instruction.

When the update instruction includes at least one of the adding instruction and the modifying instruction, the processor is configured to receive physiological characteristics information based on the update instruction; and update the second set of physiological characteristics based on the received physiological characteristics information.

The processor is also configured to receive the physiological characteristics information via a terminal or via the balance vehicle.

When comparing the first set of physiological characteristics with the second set of physiological characteristics, the processor is configured to determine whether the second set of physiological characteristics includes the first set of physiological characteristics; disable operation of the balance vehicle by the first user when it is determined that the second set of physiological characteristics does not include the first set of physiological characteristics; and enable operation of the balance vehicle by the first user when it is determined that the second set of physiological characteristics includes the first set of physiological characteristics.

Aspects of the disclosure also provide a non-transitory computer-readable medium including instructions for causing a computer to measure a first set of physiological characteristics of a first user that is currently occupying a balance vehicle; retrieve a second set of physiological characteristics from storage, wherein the second set of physiological characteristics is associated with at least one second user that is permitted to operate the balance vehicle; compare the first set of physiological characteristics with the second set of physiological characteristics; determine whether the first user is different from the second user based on the comparison; and generate a prompt that is configured to indicate whether the first user is different from the second user based on the determination.

It is to be understood that the forgoing general description and the following detailed description are illustrative and explanatory only and are not restrictive the present disclosure.

### BRIEF DESCRIPTION OF THE DRAWINGS

The accompanying drawings, which are incorporated in and constitute a part of this specification, illustrate aspects consistent with the present disclosure and, together with the description, serve to explain the principles of the present disclosure.

FIG. 1 is a flow diagram illustrating an anti-theft method for a balance vehicle according to an exemplary aspect of the present disclosure.

FIG. 2 is a flow diagram illustrating another anti-theft method for a balance vehicle according to an exemplary aspect of the present disclosure.

FIG. 3 is a flow diagram illustrating another anti-theft method for a balance vehicle according to an exemplary aspect of the present disclosure.

FIG. 4 is a flow diagram illustrating another anti-theft method for a balance vehicle according to an exemplary aspect of the present disclosure.

FIG. 5 is a flow diagram illustrating step S402 of another anti-theft method for a balance vehicle according to an exemplary aspect of the present disclosure.

FIG. 6 is a flow diagram illustrating another anti-theft method for a balance vehicle according to an exemplary aspect of the present disclosure.

FIG. 7 is a flow diagram illustrating another anti-theft method for a balance vehicle according to an exemplary aspect of the present disclosure.

FIG. 8 is a block diagram illustrating an anti-theft device for a balance vehicle according to an exemplary aspect of the present disclosure.

FIG. 9 is a block diagram illustrating a first obtaining module of an anti-theft device for a balance vehicle according to an exemplary aspect of the present disclosure.

FIG. 10 is a block diagram illustrating a first obtaining module of another anti-theft device for a balance vehicle according to an exemplary aspect of the present disclosure.

FIG. 11 is a block diagram illustrating a prompt module of another anti-theft device for a balance vehicle according to an exemplary aspect of the present disclosure.

FIG. 12 is a block diagram illustrating another anti-theft device for a balance vehicle according to an exemplary aspect of the present disclosure.

FIG. 13 is a block diagram illustrating an updating module of another anti-theft device for a balance vehicle according to an exemplary aspect of the present disclosure.

FIG. 14 is a block diagram illustrating another anti-theft device for a balance vehicle according to an exemplary aspect of the present disclosure.

FIG. 15 is a block diagram illustrating an anti-theft device for a balance vehicle according to an exemplary aspect of the present disclosure.

The specific aspects of the present disclosure, which have been illustrated by the accompanying drawings described above, will be described in detail below. These accompanying drawings and description are not intended to limit the scope of the present disclosure in any manner, but to explain the concept of the present disclosure to those skilled in the art via referencing specific aspects.

### DETAILED DESCRIPTION

Reference will now be made in detail to exemplary aspects, examples of which are illustrated in the accompanying drawings. The following description refers to the accompanying drawings in which same numbers in different drawings represent same or similar elements unless otherwise described. The implementations set forth in the following description of example aspects do not represent all implementations consistent with the present disclosure. Instead, they are merely examples of devices and methods consistent with aspects related to the present disclosure as recited in the appended claims.

The aspects of the present disclosure provide an anti-theft method for a balance vehicle. The method may be applied into a balance vehicle and other terminal devices for controlling the balance vehicle, wherein, the terminal devices may be mobile phones, computers, digital broadcasting terminals, message transceivers, game consoles, tablet devices, medical devices, fitness devices, personal digital assistants and the like.

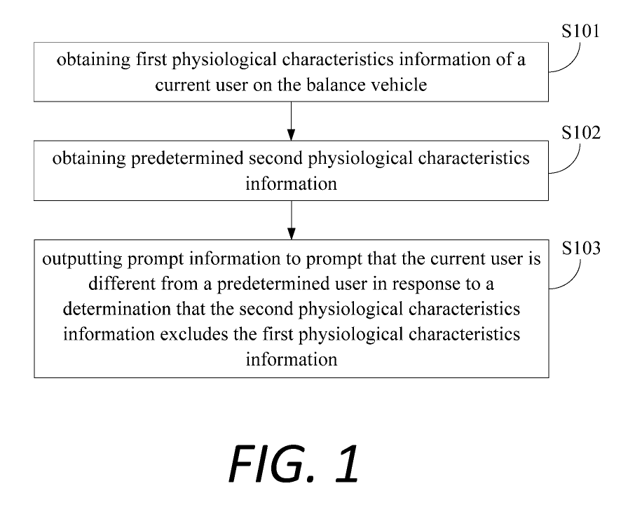

FIG. 1 is a flow diagram illustrating an anti-theft method for a balance vehicle according to an exemplary aspect. As shown in FIG. 1, the method may include steps S101 to S103.

In step S101, first physiological characteristics information (e.g., a first set of physiological characteristics) of a current user (e.g., a first user) on the balance vehicle may be obtained.

In one aspect, the first physiological characteristics information may include at least one of weight and area occupied by a foot.

In step S102, predetermined second physiological characteristics information (e.g., a second set of physiological characteristics) may be obtained from storage (e.g., memory).

The predetermined second physiological characteristics information may be predetermined physiological characteristics information of an owner of a balance vehicle or physiological characteristics information of a user associated with the owner, such as physiological characteristics information of a friend or family of the owner. Such that, the owner may prestore the physiological characteristics information of the user enabled to use the balance vehicle so as to ensure that the user may use the balance vehicle normally.

In step S103, prompt information may be outputted to prompt that the current user is different from a predetermined user in response to a determination that the second physiological characteristics information excludes the first physiological characteristics information.

In the aspect, the above technical solution may obtain first physiological characteristics information of a current user on the balance vehicle (such as weight and area occupied by a foot of the current user), obtain stored predetermined second physiological characteristics information (such as physiological characteristics information of the owner of the balance vehicle), and output prompt information to prompt the user that the current user is different form a predetermined user when the second physiological characteristics information excludes the first physiological characteristics information (namely the physiological characteristics information of the current user is not the predetermined physiological characteristics information), such that the balance vehicle may be prevented from being stolen and the property loss of the user may be avoided.

Wherein, the second physiological characteristics information may be one or more, and may belongs to one user or multiple users. When determining whether the second physiological characteristics information includes the first physiological characteristics information or not, it is expected to determine whether the first physiological characteristics information is the same as or similar to the second physiological characteristics information. Whether it is similar means whether the value of the first physiological characteristics is in an acceptable error range of the value of the second physiological characteristics, when it is in the error range, it may be determined that the second physiological characteristics information includes the first physiological characteristics information.

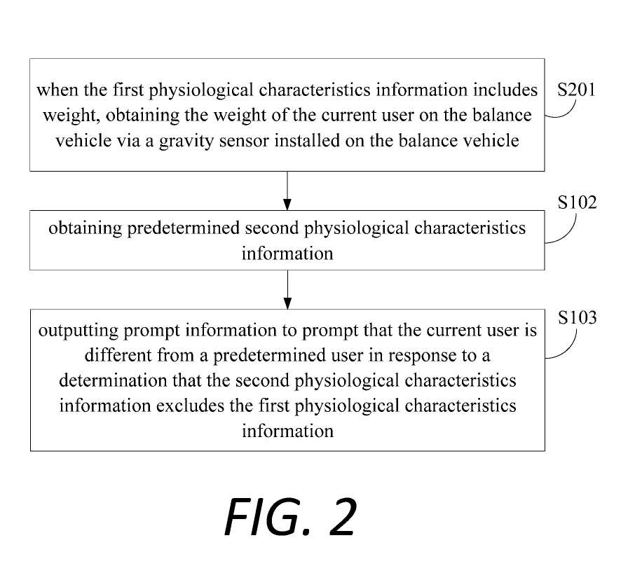

FIG. 2 is a flow diagram illustrating another anti-theft method for a balance vehicle according to an exemplary aspect. As shown in FIG. 2, when the first physiological characteristics information includes weight, the above step S101 may include step S201.

In step S201, the weight of the current user on the balance vehicle may be obtained via a gravity sensor installed on the balance vehicle.

In the aspect, the weight of the current user on the balance vehicle may be obtained via a gravity sensor installed on the bottom of the underpan of the balance vehicle. The weight of the human body may not change greatly in a short period, therefore, whether the current user is the predetermined user may be determined via the weight of the human body, such that the balance vehicle may be prevented from being stolen and the property loss of the user may be avoided.

The first physiological characteristics information may include the weight, and may also include area occupied by a foot.

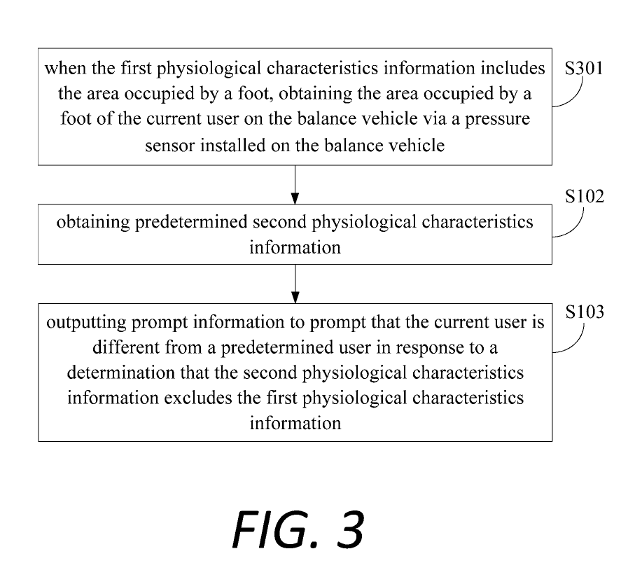

FIG. 3 is a flow diagram illustrating another anti-theft method for a balance vehicle according to an exemplary aspect. As shown in FIG. 3, when the first physiological characteristics information includes the area occupied by a foot, the above step S101 may include step S301.

In step S301, the area occupied by a foot of the current user on the balance vehicle may be obtained via a pressure sensor installed on the balance vehicle.

In the aspect, the area occupied by a foot of the current user on the balance vehicle may be obtained via a gravity sensor installed on the bottom of the underpan of the balance vehicle. The size of a foot of the human body may not change and may be recognizable, therefore, whether the current user is the predetermined user may be determined via the area occupied by a foot of the human body, such that the accuracy of the determination may be guaranteed, the balance vehicle may be prevented from being stolen, and the property loss of the user may be avoided.

Of course, in order to improve the accuracy of the determination, whether the current user is the predetermined user may be determined via the weight and the area occupied by a foot, such that prompt information may be outputted when it is determined that the current user is different from the predetermined user.

In one aspect, the outputting prompt information may include controlling the balance vehicle to output the prompt information, wherein, the prompt information may be outputted by at least one of displaying content and playing voice.

In the aspect, the prompt information may be outputted directly via the balance vehicle, such as the balance vehicle may generate an audio alarm, so that the attention of the people around the balance vehicle may be caught, which may stop the thief from stealing the balance vehicle and avoid property loss of the user.

In another aspect, the outputting prompt information may also include controlling a terminal associated with the balance vehicle to output the prompt information, wherein, the prompt information is outputted by at least one of displaying content and playing voice.

In the aspect, the terminal associated with the balance vehicle may be controlled to output the prompt information, such as the owner's terminal being bound to the balance vehicle may be controlled to output the prompt information so as to prompt the owner that the balance vehicle is stolen. For example, the prompt information may be outputted by displaying content such as a short message, and may be outputted by playing voice, the user may be informed easily.

Of course, in order to achieve better results, the balance vehicle and the terminal associated with the balance vehicle may be controlled to output the prompt information concurrently.

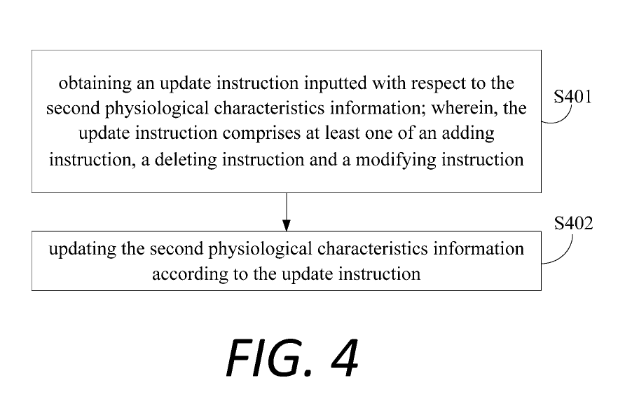

FIG. 4 is a flow diagram illustrating another anti-theft method for a balance vehicle according to an exemplary aspect. As shown in FIG. 4, in one aspect, the above method may include steps S401 to S402.

In step S401, an update instruction inputted with respect to the second physiological characteristics information may be obtained, wherein, the update instruction may include at least one of an adding instruction, a deleting instruction and a modifying instruction.

In step S402, the second physiological characteristics information may be updated according to the update instruction.

In the aspect, when the user wants to perform adding, deleting and modifying operations to the predetermined second physiological characteristics information, the update instruction with respect to the second physiological characteristics information may be inputted, such as an adding instruction, a deleting instruction and a modifying instruction, so that the second physiological characteristics information may be updated.

For example, the owner of the balance vehicle may create an account and set a login password. The predetermined second physiological characteristics information may be stored in the account. The owner may input the correct login password to login the account, and then may update the second physiological characteristics information. In this way, other people may not update the second physiological characteristics information, the security of the balance vehicle may be guaranteed.

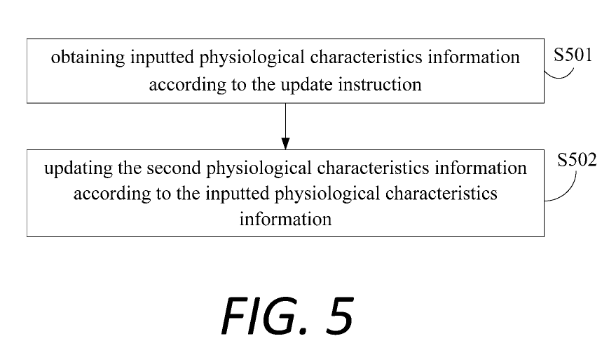

FIG. 5 is a flow diagram illustrating step S402 of another anti-theft method for a balance vehicle according to an exemplary aspect. As shown in FIG. 5, in one aspect, when the update instruction includes at least one of the adding instruction and the modifying instruction, the above step S402 may include steps S501 to S502.

In step S501, inputted physiological characteristics information may be obtained according to the update instruction.

In step S502, the second physiological characteristics information may be updated according to the inputted physiological characteristics information.

In the aspect, when the update instruction is received, the physiological characteristics information inputted by the user may be obtained. The predetermined second physiological characteristics information may be updated according to the physiological characteristics information. Such that the realtime and accuracy of the second physiological characteristics information may be guaranteed, and the accuracy of the determination that whether the balance vehicle is stolen may be guaranteed.

Wherein, when obtaining inputted physiological characteristics information, the physiological characteristics information may be obtained via any one of the followings.

Method one, the physiological characteristics information inputted via a terminal may be obtained.

In the aspect, the user may directly input the physiological characteristics information on the terminal, such as 53 kg of the weight, 370 cm² of the area occupied by feet. In this way, the user may input the physiological characteristics information on the terminal, which is very convenience.

Method two, the physiological characteristics information of a user on the balance vehicle may be obtained.

In the aspect, the physiological characteristics information of the user may be obtained via the gravity sensor and the pressure sensor installed on the balance vehicle, such as the weight and the area occupied by a foot of the human body. In this way, the accuracy of the second physiological characteristics information may be guaranteed, and the accuracy of the determination that whether the balance vehicle is stolen may be guaranteed.

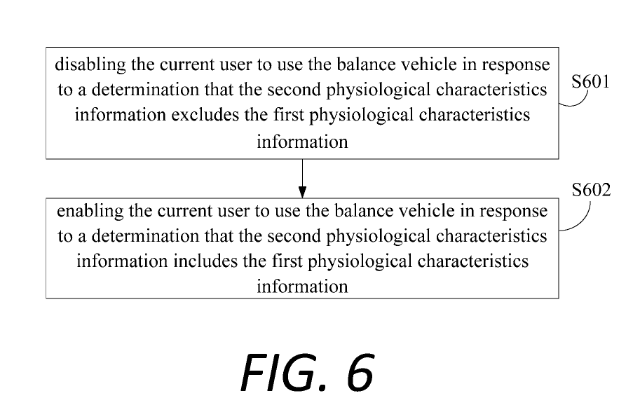

FIG. 6 is a flow diagram illustrating another anti-theft method for a balance vehicle according to an exemplary aspect. As shown in FIG. 6, in one aspect, the above method may include steps S601 to S602.

In step S601, the current user may be disabled to use the balance vehicle in response to a determination that the second physiological characteristics information excludes the first physiological characteristics information.

In step S602, the current user may be enabled to use the balance vehicle in response to a determination that the second physiological characteristics information includes the first physiological characteristics information.

In the aspect, in order to prevent the balance vehicle from being stolen, when it is determined that the second physiological characteristics information excludes the first physiological characteristics information (namely when the current user is different from the predetermined user), the current user may be disabled to use the balance vehicle, such as the balance vehicle may be locked automatically. When the second physiological characteristics information includes the first physiological characteristics information, the current user may be enabled to use the balance vehicle.

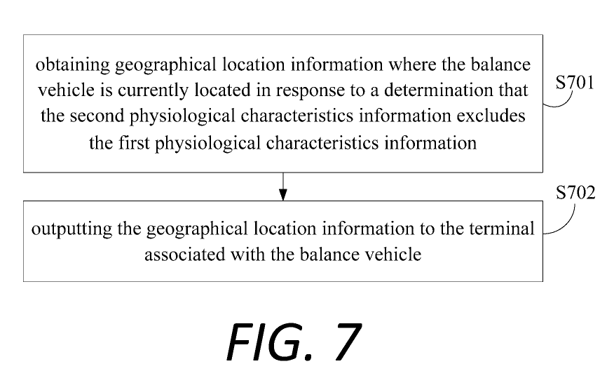

FIG. 7 is a flow diagram illustrating another anti-theft method for a balance vehicle according to an exemplary aspect. As shown in FIG. 7, in one aspect, the above method may include steps S701 to S702.

In step S701, geographical location information where the balance vehicle is currently located may be obtained in response to a determination that the second physiological characteristics information excludes the first physiological characteristics information.

In step S702, the geographical location information may be outputted to the terminal associated with the balance vehicle.

In the aspect, when it is determined that the second physiological characteristics information excludes the first physiological characteristics information (namely when the current user is different from the predetermined user), the geographical location information of the balance vehicle may be obtained, and the geographical location information may be transmitted to the terminal associated with the balance vehicle. For example, the geographical location information may be transmitted to the terminal of the owner. In this way, while the balance vehicle is stolen, the owner may find the balance vehicle according to the geographical location information, and the property loss of the user may be avoided.

The following is the device in the aspects of the present disclosure which may be used to perform the method in the aspects of the present disclosure.

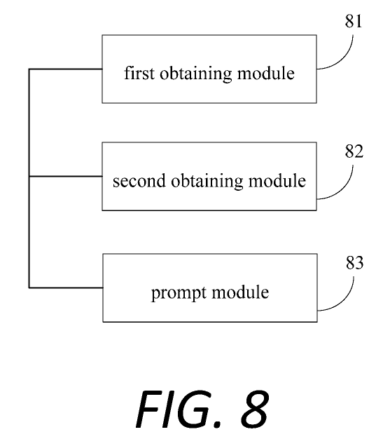

FIG. 8 is a block diagram illustrating an anti-theft device for a balance vehicle according to an exemplary aspect. The device may be implemented as the balance vehicle or the terminal device controlling the balance vehicle using software, hardware, or the combination thereof. As shown in FIG. 8, the anti-theft device for a balance vehicle may include: a first obtaining module 81 configured to obtain first physiological characteristics information of a current user on the balance vehicle; a second obtaining module 82 configured to obtain predetermined second physiological characteristics information; a prompt module configured 83 to output prompt information to prompt that the current user is different from a predetermined user in response to a determination that the second physiological characteristics information obtained by the second obtaining module excludes the first physiological characteristics information obtained by the first obtaining module.

In the aspect, the first obtaining module may obtain the first physiological characteristics information of the current user on the balance vehicle (such as weight and area occupied by a foot of the current user), the second obtaining module may obtain predetermined second physiological characteristics information (such as physiological characteristics information of the owner of the balance vehicle), the prompt module may output prompt information to prompt the user that the current user is different from a predetermined user when the second physiological characteristics information excludes the first physiological characteristics information (namely the physiological characteristics information of the current user is not the predetermined physiological characteristics information). Such that the balance vehicle may be prevented from being stolen and the property loss of the user may be avoided.

Wherein, the second physiological characteristics information may be one or more, and may belongs to one user or multiple users. When determining whether the second physiological characteristics information includes the first physiological characteristics information or not, it is expected to determine whether the first physiological characteristics information is the same as or similar to the second physiological characteristics information. Whether it is similar means whether the value of the first physiological characteristics is in an acceptable error range of the value of the second physiological characteristics, when it is in the error range, it may be determined that the second physiological characteristics information includes the first physiological characteristics information.

In one aspect, the first physiological characteristics information may include at least one of weight and area occupied by a foot.

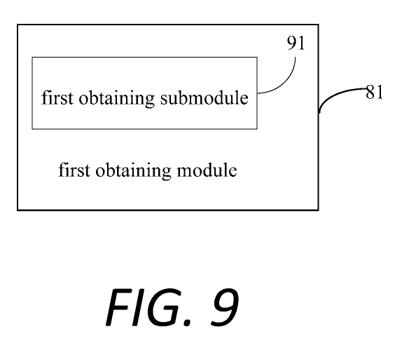

FIG. 9 is a block diagram illustrating a first obtaining module of an anti-theft device for a balance vehicle according to an exemplary aspect.

As shown in FIG. 9, in one aspect, the first physiological characteristics information may include the weight, the first obtaining module 81 may include a first obtaining submodule 91 configured to obtain the weight of the current user on the balance vehicle via a gravity sensor installed on the balance vehicle.

In the aspect, the weight of the current user on the balance vehicle may be obtained via a gravity sensor installed on the bottom of the underpan of the balance vehicle. The weight of the human body may not change greatly in a short period, therefore, whether the current user is the predetermined user may be determined via the weight of the human body, such that the balance vehicle may be prevented from being stolen and the property loss of the user may be avoided.

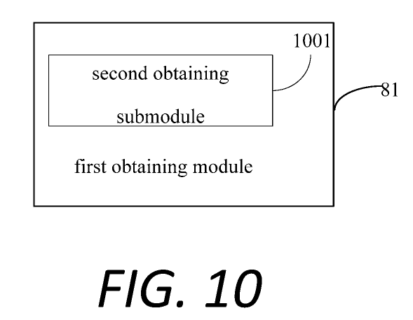

FIG. 10 is a block diagram illustrating a first obtaining module of another anti-theft device for a balance vehicle according to an exemplary aspect.

As shown in FIG. 10, in one aspect, the first physiological characteristics information may include the area occupied by a foot, the first obtaining module 81 may include a second obtaining submodule 1001 configured to obtain the area occupied by a foot of the current user on the balance vehicle via a pressure sensor installed on the balance vehicle.

In the aspect, the area occupied by a foot of the current user on the balance vehicle may be obtained via a gravity sensor installed on the bottom of the underpan of the balance vehicle. The size of a foot of the human body may not change and may be recognizable, therefore, whether the current user is the predetermined user may be determined via the area occupied by a foot of the human body, such that the accuracy of the determination may be guaranteed, the balance vehicle may be prevented from being stolen, and the property loss of the user may be avoided.

Of course, in order to improve the accuracy of the determination, whether the current user is the predetermined user may be determined via the weight and the area occupied by a foot, such that prompt information may be outputted when it is determined that the current user is different from the predetermined user.

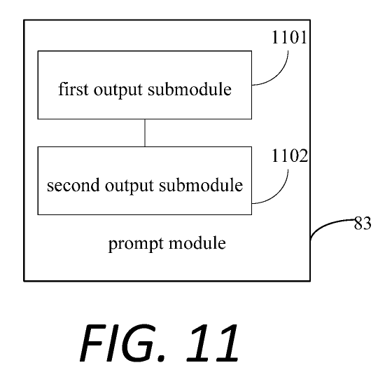

FIG. 11 is a block diagram illustrating a prompt module of another anti-theft device for a balance vehicle according to an exemplary aspect.

As shown in FIG. 11, in one aspect, the prompt module may include at least one of a first output submodule 1101 and a second output submodule 1102.

The first output submodule 1101 may be configured to control the balance vehicle to output the prompt information.

In the aspect, the prompt information may be outputted directly via the balance vehicle, such as the balance vehicle may generate an audio alarm, so that the attention of the people around the balance vehicle may be caught, which may stop the thief from stealing the balance vehicle and avoid property loss of the user.

The second output submodule 1102 may be configured to control a terminal associated with the balance vehicle to output the prompt information, wherein, the prompt information is outputted by at least one of displaying content and playing voice.

In the aspect, the terminal associated with the balance vehicle may be controlled to output the prompt information, such as the owner's terminal being bound to the balance vehicle may be controlled to output the prompt information so as to prompt the owner that the balance vehicle is stolen. For example, the prompt information may be outputted by displaying content such as a short message, and may be outputted by playing voice, the user may be informed easily.

Of course, in order to achieve better results, the balance vehicle and the terminal associated with the balance vehicle may be controlled to output the prompt information concurrently.

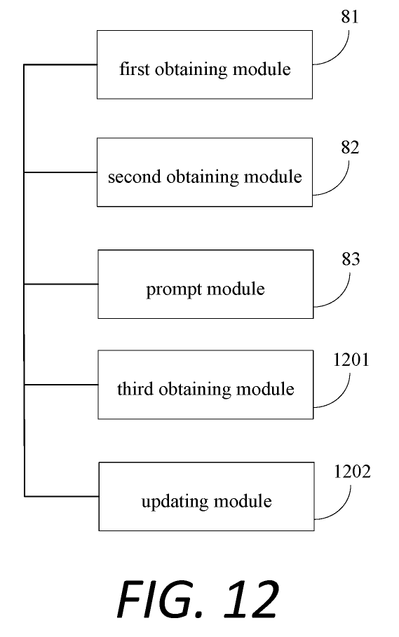

FIG. 12 is a block diagram illustrating another anti-theft device for a balance vehicle according to an exemplary aspect.

As shown in FIG. 12, in one aspect, the above device may also include a third obtaining module 1201 configured to obtain an update instruction inputted with respect to the second physiological characteristics information; an updating module 1202 configured to update the second physiological characteristics information according to the update instruction obtained by the third obtaining module; wherein, the update instruction comprises at least one of an adding instruction, a deleting instruction and a modifying instruction.

In the aspect, when the user wants to perform adding, deleting and modifying operations to the predetermined second physiological characteristics information, the update instruction with respect to the second physiological characteristics information may be inputted, such as an adding instruction, a deleting instruction and a modifying instruction, so that the second physiological characteristics information may be updated.

For example, the owner of the balance vehicle may create an account and set a login password. The predetermined second physiological characteristics information may be stored in the account. The owner may input the correct login password to login the account, and then may update the second physiological characteristics information. In this way, other people may not update the second physiological characteristics information, the security of the balance vehicle may be guaranteed.

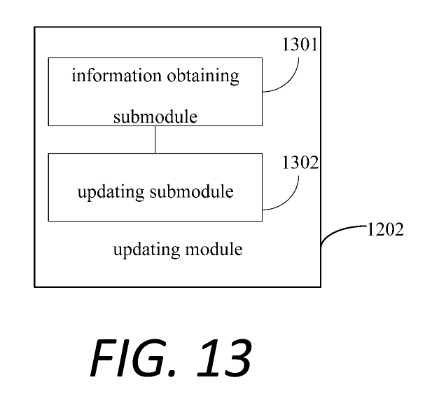

FIG. 13 is a block diagram illustrating an updating module of another anti-theft device for a balance vehicle according to an exemplary aspect.

As shown in FIG. 13, in one aspect, when the update instruction comprises at least one of the adding instruction and the modifying instruction, the updating module 1201 may include an information obtaining submodule 1301 configured to obtain inputted physiological characteristics information according to the update instruction; an updating submodule 1302 configured to update the second physiological characteristics information according to the inputted physiological characteristics information obtained by the information obtaining submodule.

In the aspect, when the update instruction is received, the physiological characteristics information inputted by the user may be obtained. The predetermined second physiological characteristics information may be updated according to the physiological characteristics information. Such that the realtime and accuracy of the second physiological characteristics information may be guaranteed, and the accuracy of the determination that whether the balance vehicle is stolen may be guaranteed.

Wherein, when obtaining inputted physiological characteristics information, the physiological characteristics information may be obtained via any one of the followings.

Method one, the physiological characteristics information inputted via a terminal may be obtained.

In the aspect, the user may directly input the physiological characteristics information on the terminal, such as 53 kg of the weight, 370 cm² of the area occupied by feet. In this way, the user may input the physiological characteristics information on the terminal, which is very convenience.

Method two, the physiological characteristics information of a user on the balance vehicle may be obtained.

In the aspect, the physiological characteristics information of the user may be obtained via the gravity sensor and the pressure sensor installed on the balance vehicle, such as the weight and the area occupied by a foot of the human body. In this way, the accuracy of the second physiological characteristics information may be guaranteed, and the accuracy of the determination that whether the balance vehicle is stolen may be guaranteed.

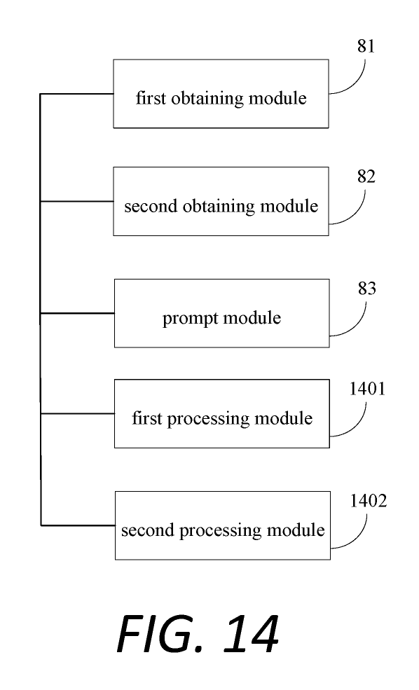

FIG. 14 is a block diagram illustrating another anti-theft device for a balance vehicle according to an exemplary aspect.

As shown in FIG. 14, in one aspect, the above device may also include a first processing module 1401 configured to disable the current user to use the balance vehicle in response to a determination that the second physiological characteristics information excludes the first physiological characteristics information; a second processing module 1402 configured to enable the current user to use the balance vehicle in response to a determination that the second physiological characteristics information includes the first physiological characteristics information.

In the aspect, in order to prevent the balance vehicle from being stolen, when it is determined that the second physiological characteristics information excludes the first physiological characteristics information (namely when the current user is different from the predetermined user), the current user may be disabled to use the balance vehicle, such as the balance vehicle may be locked automatically. When the second physiological characteristics information includes the first physiological characteristics information, the current user may be enabled to use the balance vehicle.

According to a third aspect of the aspects of the present disclosure, an anti-theft device for a balance vehicle is provided, comprising: a processor; a memory for storing processor-executable instructions; wherein the processor is configured to: obtain first physiological characteristics information of a current user on the balance vehicle; obtain predetermined second physiological characteristics information; output prompt information to prompt that the current user is different form a predetermined user in response to a determination that the second physiological characteristics information excludes the first physiological characteristics information.

The above processor may further be configured such that the first physiological characteristics information comprises at least one of weight and area occupied by a foot.

The above processor may further be configured such that the first physiological characteristics information comprises the weight; the obtain first physiological characteristics information of a current user on the balance vehicle comprises: obtain the weight of the current user on the balance vehicle via a gravity sensor installed on the balance vehicle.

The above processor may further be configured such that the first physiological characteristics information comprises the area occupied by a foot; the obtain first physiological characteristics information of a current user on the balance vehicle comprises: obtain the area occupied by a foot of the current user on the balance vehicle via a pressure sensor installed on the balance vehicle.

The above processor may further be configured such that the output prompt information comprises at least one of: control the balance vehicle to output the prompt information; control a terminal associated with the balance vehicle to output the prompt information; wherein, the prompt information is outputted by at least one of displaying content and playing voice.

The above processor may further be configured to obtain an update instruction inputted with respect to the second physiological characteristics information; update the second physiological characteristics information according to the update instruction; wherein, the update instruction comprises at least one of an adding instruction, a deleting instruction and a modifying instruction.

The above processor may further be configured such that when the update instruction comprises at least one of the adding instruction and the modifying instruction, the update the second physiological characteristics information according to the update instruction comprises: obtain inputted physiological characteristics information according to the update instruction; update the second physiological characteristics information according to the inputted physiological characteristics information.

The above processor may further be configured such that the obtain inputted physiological characteristics information comprises: obtain the physiological characteristics information inputted via a terminal; or obtain the physiological characteristics information of a user on the balance vehicle.

The above processor may further be configured to disable the current user to use the balance vehicle in response to a determination that the second physiological characteristics information excludes the first physiological characteristics information; enable the current user to use the balance vehicle in response to a determination that the second physiological characteristics information includes the first physiological characteristics information.

The above processor may further be configured to obtain geographical location information where the balance vehicle is currently located in response to a determination that the second physiological characteristics information excludes the first physiological characteristics information; output the geographical location information to the terminal associated with the balance vehicle.

The above processor may further be configured to obtain geographical location information where the balance vehicle is currently located in response to a determination that the second physiological characteristics information excludes the first physiological characteristics information; output the geographical location information to the terminal associated with the balance vehicle.

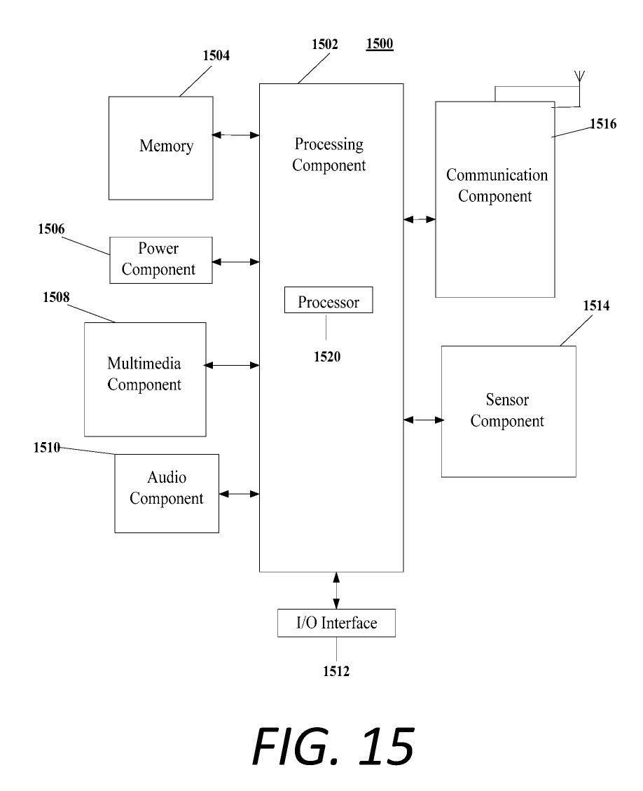

FIG. 15 is a block diagram illustrating an anti-theft device for a balance vehicle according to an exemplary aspect. For example, the device 1500 may be a mobile phone, a computer, a digital broadcast terminal, a messaging device, a gaming console, a tablet, a medical device, an exercise equipment, a personal digital assistant, and the like.

The device 1500 may include one or more of the following components: a processing component 1502, a memory 1504, a power component 1506, a multimedia component 1508, an audio component 1510, an input/output (I/O) interface 1512, a sensor component 1514, and a communication component 1516.

The processing component 1502 typically controls overall operations of the device 1500, such as the operations associated with display, data communications, and recording operations. The processing component 1502 may include one or more processors 1520 to execute instructions to perform all or part of the steps in the above described methods. Moreover, the processing component 1502 may include one or more modules which facilitate the interaction between the processing component 1502 and other components. For instance, the processing component 1502 may include a multimedia module to facilitate the interaction between the multimedia component 1508 and the processing component 1502.

The memory 1504 is configured to store various types of data to support the operation of the device 1500. Examples of such data include instructions for any applications or methods operated on the device 1500, environmental states, times, etc. The memory 1504 may be implemented using any type of volatile or non-volatile memory devices, or a combination thereof, such as a static random access memory (SRAM), an electrically erasable programmable read-only memory (EEPROM), an erasable programmable read-only memory (EPROM), a programmable read-only memory (PROM), a read-only memory (ROM), a magnetic memory, a flash memory, a magnetic or optical disk.

The power component 1506 provides power to various components of the device 1500. The power component 1506 may include a power management system, one or more power sources, and any other components associated with the generation, management, and distribution of power in the device 1500.

The multimedia component 1508 includes a screen providing an output interface between the device 1500 and the user. In some aspects, the screen may include a liquid crystal display (LCD) and a touch panel (TP). If the screen includes the touch panel, the screen may be implemented as a touch screen to receive input signals from the user. The touch panel includes one or more touch sensors to sense touches, swipes, and gestures on the touch panel. The touch sensors may not only sense a boundary of a touch or swipe action, but also sense a period of time and a pressure associated with the touch or swipe action.

The audio component 1510 is configured to output and/or input audio signals. For example, the audio component 1510 includes a microphone (“MIC”) configured to receive an external audio signal when the device 1500 is in an operation mode, such as a recording mode and a voice recognition mode. The received audio signal may be further stored in the memory 1504 or transmitted via the communication component 1516. In some aspects, the audio component 1510 further includes a speaker to output audio signals.

The I/O interface 1512 provides an interface between the processing component 1502 and peripheral interface modules, such as a keyboard, a click wheel, buttons, and the like. The buttons may include, but are not limited to, a home button, a volume button, a starting button, and a locking button.

The sensor component 1514 includes one or more sensors to provide status assessments of various aspects of the device 1500. For instance, the sensor component 1514 may detect an open/closed status of the device 1500, relative positioning of components, e.g., the display and the keypad, of the device 1500, a change in position of the device 1500 or a component of the device 1500, a presence or absence of user contact with the device 1500, an orientation or an acceleration/deceleration of the device 1500, and a change in temperature of the device 1500. The sensor component 1514 may include a proximity sensor configured to detect the presence of nearby objects without any physical contact. The sensor component 1514 may also include a light sensor, such as a CMOS or CCD image sensor, for use in imaging applications. In some aspects, the sensor component 1514 may also include an accelerometer sensor, a gyroscope sensor, a magnetic sensor, a pressure sensor, or a temperature sensor.

The communication component 1516 is configured to facilitate communication, wired or wirelessly, between the device 1500 and other devices. The device 1500 can access a wireless network based on a communication standard, such as WiFi, 2G, or 3G, or a combination thereof. In one example aspect, the communication component 1516 receives a broadcast signal or broadcast associated information from an external broadcast management system via a broadcast channel. In one example aspect, the communication component 1516 further includes a near field communication (NFC) module to facilitate short-range communications. For example, the NFC module may be implemented based on a radio frequency identification (RFID) technology, an infrared data association (IrDA) technology, an ultra-wideband (UWB) technology, a Bluetooth (BT) technology, and other technologies.

In example aspects, the device 1500 may be implemented with one or more application specific integrated circuits (ASICs), digital signal processors (DSPs), digital signal processing devices (DSPDs), programmable logic devices (PLDs), field programmable gate arrays (FPGAs), controllers, micro-controllers, microprocessors, or other electronic components, for performing the above described methods.

In example aspects, there is also provided a non-transitory computer readable storage medium including instructions, such as included in the memory 1504, executable by the processor 1520 in the device 1500, for performing the above-described methods. For example, the non-transitory computer-readable storage medium may be a ROM, a random access memory (RAM), a CD-ROM, a magnetic tape, a floppy disc, an optical data storage device, and the like.

A non-transitory computer readable storage medium having stored therein instructions that, when executed by the processor of the device 1500, causes the device 1500 to perform the above described anti-theft method for a balance vehicle, comprising: obtaining first physiological characteristics information of a current user on the balance vehicle; obtaining predetermined second physiological characteristics information; outputting prompt information to prompt that the current user is different from a predetermined user in response to a determination that the second physiological characteristics information excludes the first physiological characteristics information.

In one aspect, the first physiological characteristics information comprises at least one of weight and area occupied by a foot.

In one aspect, the first physiological characteristics information comprises the weight; the obtaining first physiological characteristics information of a current user on the balance vehicle comprises: obtaining the weight of the current user on the balance vehicle via a gravity sensor installed on the balance vehicle.

In one aspect, the first physiological characteristics information comprises the area occupied by a foot; the obtaining first physiological characteristics information of a current user on the balance vehicle comprises: obtaining the area occupied by a foot of the current user on the balance vehicle via a pressure sensor installed on the balance vehicle.

In one aspect, the outputting prompt information comprises at least one of: controlling the balance vehicle to output the prompt information; controlling a terminal associated with the balance vehicle to output the prompt information; wherein, the prompt information is outputted by at least one of displaying content and playing voice.

In one aspect, the method further comprising: obtaining an update instruction inputted with respect to the second physiological characteristics information; updating the second physiological characteristics information according to the update instruction; wherein, the update instruction comprises at least one of an adding instruction, a deleting instruction and a modifying instruction.

In one aspect, when the update instruction comprises at least one of the adding instruction and the modifying instruction, the updating the second physiological characteristics information according to the update instruction comprises: obtaining inputted physiological characteristics information according to the update instruction; updating the second physiological characteristics information according to the inputted physiological characteristics information.

In one aspect, the obtaining inputted physiological characteristics information comprises: obtaining the physiological characteristics information inputted via a terminal; or obtaining the physiological characteristics information of a user on the balance vehicle.

In one aspect, the method further comprising: disabling the current user to use the balance vehicle in response to a determination that the second physiological characteristics information excludes the first physiological characteristics information; enabling the current user to use the balance vehicle in response to a determination that the second physiological characteristics information includes the first physiological characteristics information.

In one aspect, the method further comprising: obtaining geographical location information where the balance vehicle is currently located in response to a determination that the second physiological characteristics information excludes the first physiological characteristics information; outputting the geographical location information to the terminal associated with the balance vehicle.

In one aspect, the method further comprising: obtaining geographical location information where the balance vehicle is currently located in response to a determination that the second physiological characteristics information excludes the first physiological characteristics information; outputting the geographical location information to the terminal associated with the balance vehicle.

It is noted that the various modules, sub-modules, units, and components in the present disclosure can be implemented using any suitable technology. For example, a module may be implemented using circuitry, such as an integrated circuit (IC). As another example, a module may be implemented as a processing circuit executing software instructions.

Other aspects of the invention will be apparent to those skilled in the art from consideration of the specification and practice of the disclosures herein. This application is intended to cover any variations, uses, or adaptations of the disclosure following the general principles thereof and including such departures from the present disclosure as come within known or customary practice in the art. It is intended that the specification and aspects be considered as illustrative only, with a true scope and spirit of the disclosure being indicated by the following claims.

It will be appreciated that the present disclosure is not limited to the exact construction that has been described above and illustrated in the accompanying drawings, and that various modifications and changes can be made without departing from the scope thereof. It is intended that the scope of the invention only be limited by the appended claims.

What is claimed is:

1. An anti-theft method for a balance vehicle, comprising:
measuring a first set of physiological characteristics of a first user that is currently occupying the balance vehicle;
retrieving a second set of physiological characteristics from storage, wherein the second set of physiological characteristics is associated with at least one second user that is permitted to operate the balance vehicle;
comparing the first set of physiological characteristics with the second set of physiological characteristics;
determining whether the first user is different from the second user based on the comparison; and
generating a prompt that is configured to indicate whether the first user is different from the second user based on the determination.

2. The method of claim 1, wherein the first set of physiological characteristics includes at least one of a weight of the first user and an area occupied by a foot of the first user.

3. The method of claim 2, wherein the first set of physiological characteristics includes the weight of the first user, and wherein the weight of the first user is measured via a weight sensor installed on the balance vehicle.

4. The method of claim 2, wherein the first set of physiological characteristics includes the area occupied by the foot of the first user, and wherein the area is measured via a pressure sensor installed on the balance vehicle.

5. The method of claim 1, wherein the prompt is generated by at least one of displaying content and playing voice.

6. The method of claim 1, further comprising:
receiving an update instruction with respect to the second set of physiological characteristics; and
updating the second set of physiological characteristics based on the update instruction, wherein the update instruction includes at least one of an adding instruction, a deleting instruction, and a modifying instruction.

7. The method of claim 6, wherein when the update instruction includes at least one of the adding instruction and the modifying instruction, updating the second set of physiological characteristics includes:
receiving physiological characteristics information based on the update instruction; and
updating the second set of physiological characteristics based on the received physiological characteristics information.

8. The method of claim 7, wherein the physiological characteristics information is received via a terminal or via the balance vehicle.

9. The method of claim 1, wherein comparing the first set of physiological characteristics with the second set of physiological characteristics includes determining whether the second set of physiological characteristics includes the first set of physiological characteristics, and wherein the method further comprises:
disabling operation of the balance vehicle by the first user when it is determined that the second set of physiological characteristics does not include the first set of physiological characteristics; and
enabling operation of the balance vehicle by the first user when it is determined that the second set of physiological characteristics includes the first set of physiological characteristics.

10. An anti-theft device for a balance vehicle, comprising:
a processor;
a memory configured to store instructions executable by the processor;
wherein the processor is configured to:
measure a first set of physiological characteristics of a first user that is currently occupying the balance vehicle;
retrieve a second set of physiological characteristics from the memory, wherein the second set of physiological characteristics is associated with at least one second user that is permitted to operate the balance vehicle;
compare the first set of physiological characteristics with the second set of physiological characteristics;
determine whether the first user is different from the second user based on the comparison; and
generate a prompt that is configured to indicate whether the first user is different from the second user based on the determination.

11. The device of claim 10, wherein the first set of physiological characteristics includes at least one of a weight of the first user and an area occupied by a foot of the first user.

12. The device of claim 11, wherein the first set of physiological characteristics includes the weight of the first user, and wherein the weight of the first user is measured via a weight sensor installed on the balance vehicle.

13. The device of claim 11, wherein the first set of physiological characteristics includes the area occupied by the foot of the first user, and wherein the area is measured via a pressure sensor installed on the balance vehicle.

14. The device of claim 10, wherein the processor is further configured to generate the prompt by at least one of displaying content and playing voice.

15. The device of claim 10, the processor is further configured to:
receive an update instruction with respect to the second set of physiological characteristics; and
update the second set of physiological characteristics based on the update instruction, wherein the update instruction includes at least one of an adding instruction, a deleting instruction, and a modifying instruction.

16. The device of claim 15, wherein when the update instruction includes at least one of the adding instruction and the modifying instruction, the processor is further configured to:
receive physiological characteristics information based on the update instruction; and
update the second set of physiological characteristics based on the received physiological characteristics information.

17. The device of claim 16, wherein the processor is further configured to receive the physiological characteristics information via a terminal or via the balance vehicle.

18. The device of claim 10, wherein when comparing the first set of physiological characteristics with the second set of physiological characteristics, the processor is further configured to:
determine whether the second set of physiological characteristics includes the first set of physiological characteristics;
disable operation of the balance vehicle by the first user when it is determined that the second set of physiological characteristics does not include the first set of physiological characteristics; and
enable operation of the balance vehicle by the first user when it is determined that the second set of physiological characteristics includes the first set of physiological characteristics.

19. A non-transitory computer-readable medium comprising instructions for causing a computer to:
measure a first set of physiological characteristics of a first user that is currently occupying a balance vehicle;
retrieve a second set of physiological characteristics from storage, wherein the second set of physiological characteristics is associated with at least one second user that is permitted to operate the balance vehicle;
compare the first set of physiological characteristics with the second set of physiological characteristics;
determine whether the first user is different from the second user based on the comparison; and
generate a prompt that is configured to indicate whether the first user is different from the second user based on the determination.
